(19) United States
(12) Patent Application Publication
(10) Pub. No.: US 2026/0260276 A1
(43) Pub. Date: Sep. 3, 2026

(54) System and Method for Matching Pets with Pet Products

(71) Applicant: Chewy, Inc., Plantation, FL (US)

(72) Inventors: Matthew Joseph Hornung, Plymouth, MN (US); Vamsi Krishna Thatikonda, Snoqualmie, WA (US); Sathappan Ramaswamy, Plymouth, MN (US)

(21) Appl. No.: 18/880,100

(22) PCT Filed: Aug. 15, 2024
(86) PCT No.: PCT/US2024/042458

§ 371 Date: Dec. 30, 2024

Related U.S. Application Data
(60) Provisional application No. 63/519,943, filed on Aug. 16, 2023.

(51) Int. Cl. G06Q 30/0601; G06V 30/19

(52) U.S. Cl. CPC G06Q 30/0631; G06Q 30/0625; G06V 30/19007

(57) ABSTRACT

A method of matching a pet with a pet product includes, at a server system including one or more processors, generate pet-product relationship data by comparing product content information associated with a product to pet information stored in a pet profile database, and cause a user interface to update a rendering of the product to include a rendering of information associated with the pet-product relationship data.

### CROSS-REFERENCE TO RELATED APPLICATIONS

This application claims the benefit of U.S. Provisional Patent Application No. 63/519,943 filed Aug. 16, 2023 entitled “System and Method for Matching Pets with Pet Products”, which is incorporated by reference herein in its entirety.

### TECHNICAL FIELD

The present disclosure generally relates to systems and methods for matching pets with pet products and, in some embodiments, to a system and method for matching pet data including characteristics of a pet to pet product data for a plurality of different pet products.

### SUMMARY

In one embodiment there is a method of matching a pet with a pet product including at a server system including one or more processors, generating pet-product relationship data by comparing product content information associated with a product to pet information stored in a pet profile database, and causing a user interface to update a rendering of the product to include a rendering of information associated with the pet-product relationship data.

In some embodiments, the method further includes at the server system including one or more processors: accessing a scanned image file from a digital image of a product, and running an optical character recognition utility on the scanned image file to determine the product content information associated with the product, and the comparing the product content information associated with a product with pet information is based on the determined product content information. In some embodiments, the method further includes at the server system including one or more processors, preventing the user interface from displaying product purchase information associated with the product based on the pet-product relationship data.

In some embodiments, the method further includes identifying mismatch data in response to receiving a request via the user interface to display product purchase information based on a product query and the pet-product relationship data, and restricting display of at least one product based on the mismatch data. In some embodiments, the method further includes at the server system including one or more processors: receiving a product request, via the user interface, to display available products based on a product query, determining that the product request is not associated with a particular pet, prior to rendering, on the user interface, product information based on the product query, directing the user interface to render a pet information request, receiving, via the user interface, pet identification information based on the pet information request, based on the product query and the pet identification information, identifying pet-product relationship data, and directing the user interface to display product information based on the product query and the pet-product relationship data.

In some embodiments, the method further includes, at the server system including one or more processors, causing an applicator device to apply a pet-specific label to a product package prior to shipment by a fulfillment center, and the pet-specific label is based on the pet-product relationship data. In some embodiments, the information associated with the pet-product relationship data includes product recommendation information. In some embodiments, the pet-product relationship data comprises at least one of breed-specific pet information; allergy information for a specific pet; ingredient compatibility information with a specific pet; product compatibility information with a specific pet; ingredient dislike information for a specific pet; and product dislike information for a specific pet.

In some embodiments, the product content information includes at least one of product ingredient information; product allergy information; unity price information; and information not contained within product package indicia. In some embodiments, the optical character recognition utility includes a text similarity algorithm and matching algorithm configured to identify an exact match for predetermined attributes. In some embodiments, the method further includes, at the server system including one or more processors, directing the user interface to display a supplied product image and product content information that is not displayed on the supplied product image. In some embodiments, the method further includes, at the server system including one or more processors, directing the user interface to display unit price comparison data for all products responsive to a product query based on the pet-product relationship data.

In some embodiments, the method further includes, at the server system including one or more processors, directing the user interface to display diet recommendation information based on the pet-product relationship data. In some embodiments, the product content information includes a parsed list of ingredients corresponding to the product and the pet information includes pet allergy data and pet dietary preference data, and the pet-product relationship data is indicative of an expected interaction with the product based on a comparison of the parsed list of ingredients to the pet allergy data and pet dietary preference data.

In another embodiment, there is a system including one or more processors and memory storing one or more programs to be executed by the one or more processors, the one or more programs including instructions for executing one or more of the operations included in the method described above.

In another embodiment, there is a non-transitory computer readable storage medium storing one or more programs configured for execution by a computer system, the one or more programs including instructions for executing one or more of the operations included in the method described above.

### BRIEF DESCRIPTION OF THE DRAWINGS

The following detailed description of embodiments of the system and method for matching pets with pet products, will be better understood when read in conjunction with the appended drawings of an exemplary embodiment. It should be understood, however, that the invention is not limited to the precise arrangements and instrumentalities shown.

In the drawings:

FIG. 1 is a block diagram illustrating an implementation of a system for automatically matching pets with pet products in accordance with an exemplary embodiment of the present disclosure;

FIG. 2 is a flowchart diagram illustrating an implementation of the system of FIG. 1 determining product content information;

FIG. 3 illustrates an exemplary user interface prior to executing an exemplary method of the present disclosure;

FIG. 4 illustrates an exemplary user interface following the execution of the method of the present disclosure referenced in FIG. 3;

FIG. 5 illustrates another exemplary user interface including examples of renderings of pet-product relationship data;

FIG. 6 illustrates another exemplary user interface including examples of renderings of pet-product relationship data;

FIG. 7 illustrates another exemplary user interface including examples of renderings of pet-product relationship data; and

FIG. 8 illustrates an exemplary flow chart for a method of matching pets with pet products according to an embodiment of the present disclosure.

### DETAILED DESCRIPTION

There exists a variety of different pet products that a consumer may purchase via online marketplaces and/or brick and mortar stores. Those pet products are comprised of a plurality of different elements that, for a specific pet, may come with positive and/or negative interactions. For example, pet foodstuffs products (e.g., pet food, a dry dog food, wet cat food) are often comprised of numerous ingredients, some of which a consumer's pet may be allergic to. In order to ensure the health, safety, and/or happiness of their pet, a consumer may want to know the elements/ingredients to a pet product prior to purchasing it. However, doing so can be very time consuming and overwhelming given the quantity of products to choose from and the variety of elements and ingredients that make up those products. Furthermore, a pet's size, breed, age and/or weight may contribute to conflicts in ways that a consumer is not aware.

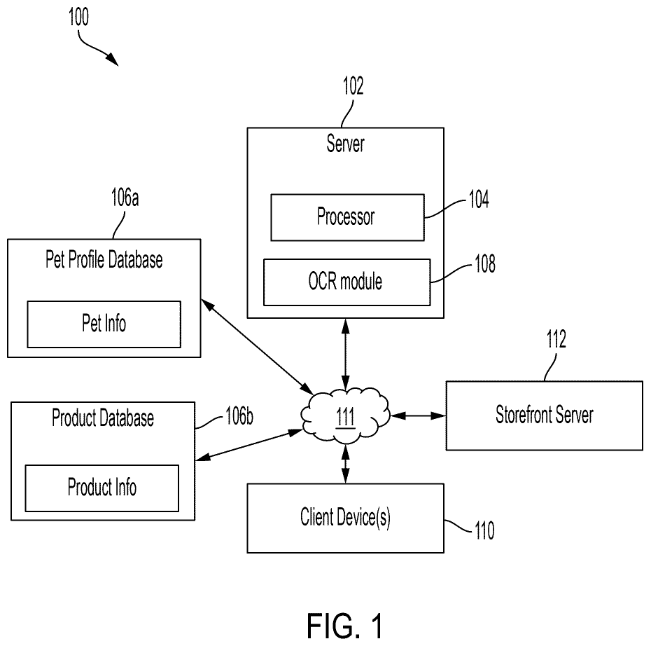

Referring to the drawings in detail, wherein like reference numerals indicate like elements throughout, there is shown in FIG. 1 a system for matching pets with pet products, generally designated 100 and referred to as system 100 herein, in accordance with an exemplary embodiment of the present invention. The system 100 may be configured to automatically compare information for pet products to information about a pet to determine a pet-product relationship data and render at a user interface that pet-product relationship data. In some embodiments, the system 100 may be configured to determine and render the pet-product relationship data for a plurality of different products displayed for purchase at an online storefront. In some embodiments, the system 100 may be configured to enable a consumer to quickly and accurately determine from the rendered pet-product relationship data, which pet products are preferable, non-preferable, safe, and/or unsafe for their pet. For sake of brevity, aspects of the present disclosure will be described with reference to pet products that are foodstuffs (e.g., dog food, cat food, fish food) and those products interactions with a pet. However, pet products may include any product intended for or suitable for use with a pet such as, but not limited to, foodstuffs, over the counter medications, prescription medications, hygiene products, topical ointments/medications, toys, clothing, harnesses, electric fences or any other product having particular materials/ingredients or effects that a pet may have an adverse reaction to. In some embodiments, pet products may further include products not explicitly intended for use with a pet, but to which the pet may have an adverse reaction. For example, certain flora is known to be toxic or harmful to different breeds of pet (e.g., lilies are toxic to cats).

In some embodiments, the system 100 is configured to match consumers with products for purchase. For example, the system 100 is not limited to pets and pet products. In some instances, the system 100 is configured to match human consumers with foodstuffs, toys, clothes, topical ointments, prescription medications, over the counter medications, hygiene products, and/or other products intended for use with human beings in generally the same manner as discussed herein with reference to pets and pet products. For sake of brevity, the system 100 and methods of the present disclosure will be described with reference to pets and pet products.

In one embodiment, the system 100 includes one or more computers having one or more processors and memory (e.g., one or more nonvolatile storage devices). In some embodiments, memory or computer readable storage medium of memory stores programs, modules and data structures, or a subset thereof for a processor to control and run the various systems and methods disclosed herein. In one embodiment, a non-transitory computer readable storage medium having stored thereon computer-executable instructions which, when executed by a processor, perform one or more of the methods disclosed herein.

There is shown in FIG. 1 a block diagram illustrating an implementation of the system 100. While some example features are illustrated, various other features have not been illustrated for the sake of brevity and so as not to obscure pertinent aspects of the embodiments disclosed herein. In some embodiments, the system 100 may alternatively be referred to as a server system 100. The system 100 may include a server 102 in communication with a pet profile database 106a, product database 106b, a storefront server 112 and at least one client device 110 via a network 111. The server 102 may include one or more processors 104 and/or an optical character recognition (OCR) utility 108 configured to extract text data from a digital image including text. The OCR utility 108 may be configured to receive one or more digital images of a product and determine product content information associated with the product.

Network 111 may be representative of any suitable type, including, but not limited to, individual connections via the Internet, such as cellular or Wi-Fi networks. In some embodiments, network 105 may connect terminals, services, computing devices, external devices using direct connections, such as, but not limited to, radio frequency identification (RFID), near-field communications (NFC), Bluetooth™, low-energy Bluetooth™ (BLE), Wi-Fi™, Zigbee™, ambient backscatter communication (ABC) protocols, USB, WAN, or LAN. Because the information transmitted may be personal or confidential, security concerns may dictate one or more of these types of connection be encrypted or otherwise secured. In some embodiments, however, the information being transmitted may be less personal, and therefore, the network connections may be selected for convenience over security.

Network 111 may include any type of computer networking arrangement used to exchange data. For example, network 105 may be representative of the Internet, a private data network, virtual private network using a public network and/or other suitable connection(s) that enables components in system 100 to send and receive information between the components of system 100.

The server 102 may be configured to match pets to pet products offered for sale at an online storefront generated by the storefront server 112. The storefront server 112 may be, for example, a single server or a collection of networked servers configured to generate and transmit to a client device 110 an online storefront at which one or more products may be purchased. The online storefront may be a customer facing user-interface (UI) having rendered thereon interactable graphical user interface (GUI) elements corresponding to products for purchase. For example, the customer facing UI may include interactable GUI elements representative of pet-products, such as foodstuffs, that a user may interact with, via a client device 110, to view and/or purchase said pet-product. The storefront server 112 may be accessible to client devices 110 via network 111 such that users (e.g., pet owners) may access the customer facing UI via their client device 110 to browse and/or purchase products and services available at the online storefront. Client device 110 may be any networked computing device such as, but not limited to, desktop computers, laptops, smart phones, and/or tablets.

The pet profile database 106a may have stored thereon pet information and, in some instances, pet owner information. Pet information stored on the pet profile database 106a may include data indicating one or more of: a pet's age, a pet's name, a pet's species, a pet's breed, a pet's medical record or portions thereof, a pet's hereditary information, a pet's allergies, a pet's dietary preferences (e.g., flavor profile likes/dislikes), a pet's non-dietary preferences (e.g., other likes and dislikes), a pet's height, a pet's weight, a pet's color and/or other physical characteristics of pet. Pet profile database 106a may have stored thereon pet information for a plurality of different pets. Pet information stored on the pet profile database 106a relating to pet allergies may include data indicating one or more foreign substances (e.g., pollens, foods, medications, man-made and/or naturally occurring materials) that a pet is allergic to. For example, pet information for a dog named Chopper stored in the pet profile database 106a may indicate that Chopper is allergic to wheat gluten. Pet information stored on the pet profile database 106a relating to pet dietary preferences may include data indicating one or more flavor profiles, foodstuffs or ingredients thereof that a pet likes and/or dislikes. For example, pet information for the dog Chopper may include an indication that Chopper likes peanut butter and dislikes pineapple.

The pet profile database 106a may be populated with pet information via one or more sources such as, but not limited to, pet owner devices, veterinarian systems, external databases storing pet medical records, smart pet appliances, and pet wearable devices. For example, pet information such as a pet's likes, dislikes, and allergies with regards to pet foodstuffs may be uploaded to the pet profile database 106a via client device 110. The pet information related to a pet may be associated with the pet owner information for the owner of that pet that is also stored on the pet profile database 106a.

The product database 106b may have stored thereon product content information relating to one or more products offered for sale at the online storefront generated by the storefront server 112. In some embodiments, there is product content information stored on the product database 106b for a plurality of different products. For each product having product content information stored on the product database 106b, the product content information may include, for example, product type (e.g., dog food, cat food), product name, product brand, unit pricing, digital images of the product, product weight, intended use instructions, usage restrictions, storage requirements, expiration dates, surfaces treatments, approved standards (e.g., FDA approved foodstuff), and whether the product is approved for human consumption. In some instances, product content information stored on the database 106b for a product may include product composition data such as, but not limited to, a listing of one or more ingredients or materials included therein. For example, if the product is a dog food, the product content information may include that the ingredients for the dog food are chicken, rice flour, corn gluten, dried peas, soybean meal and vegetable juice. As a further example, if the product is a pet bed, the product content information may include that materials included in the bed are synthetic fabrics, faux fur, and/or polyester. In some embodiments, the pet profile database 106a and product database 106b may be a single database storing both pet information and product content information.

In some embodiments, the source of product data in product database 106b may include a digital image. For example, a product entry in product database 106b may be missing some or all product composition data (e.g., ingredients for pet food). Server 102 may be configured to populate database 106b with the missing data using a digital image associated with the product such as an image of the product package. The digital image may be stored in database 106b, for example.

Server 102 may be configured with a utility (e.g., an OCR utility) that extracts composition information from the digital image and populates product database 106b with the extracted information. In some embodiment, product database 106b includes some, but not all, composition information associated with the product. Server 102 may be configure with a utility that extracts all composition information from the digital image and then selected populates database 106b with any missing composition information. For example, at the time product content information for a particular dog food is populated on the database 106b it includes the name of the dog food, the price of the dog food and images of the packaging the dog food is sold in. Further to this example, in at least one of the images of the packaging there is illustrated an ingredients label printed on the package that contains product composition data (e.g., the ingredients that make up the dog food). Accordingly, in this example the product content information includes the product composition data via the digital image of the packaging, however the product composition data is not in a machine-readable text format.

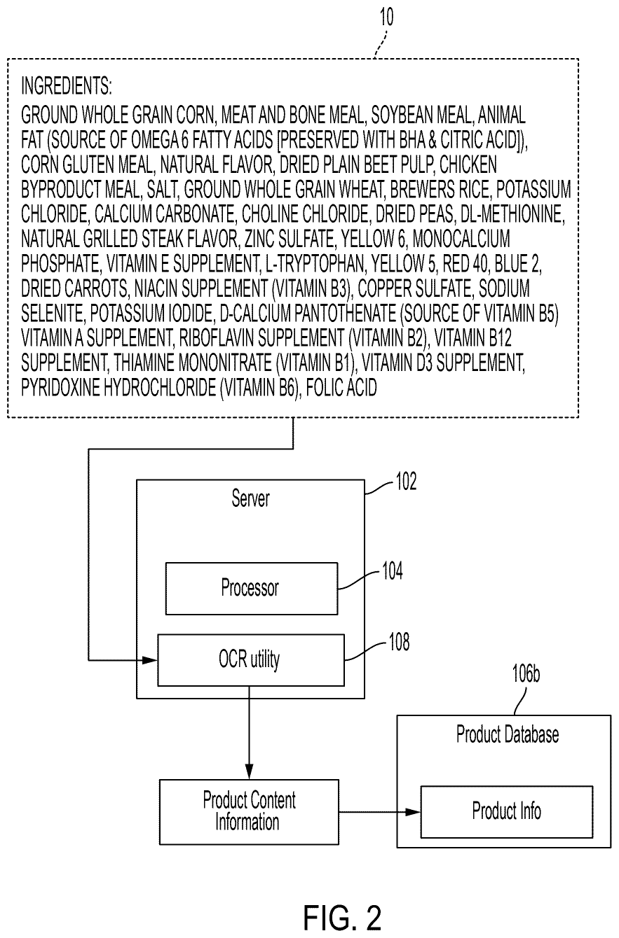

Referring to FIG. 2, in some embodiments the system 100 is configured to determine product content information based on a digital image of a product. In some embodiments, the digital image may be an image of a product packaging, a portion thereof, a label applied thereto, and/or marketing material related to the product. Product content information ascertainable from the digital image may include, but is not limited to, ingredient information, nutritional information, intended use information, net weight, package dimensions, and other information not readily ascertainable from commercial bar code databases. The system 100 may be configured to receive a digital image of a product, or a scanned image file thereof, and execute optical character recognition on the digital image. In some instances, digital images of products are directly received at the server 102 (e.g., from a manufacturer of the product) and include a reference to a side or surface of the product to which the digital image corresponds. For example, a digital image received at the server 102 includes an indication that the digital image is of the front of the product 102. In some instances, the digital image is generated via an image capture device (e.g., a camera). For example, the physical product may be received and a user may generate one or more digital images via a camera, which are then sent to the server 102. In further embodiments, the digital image is generated by placing the physical product into a scanning device. For example, a physical product is placed into a scanning device and the scanning device automatically generates digital images from at least five different sides of the product, which are transmitted to the server 102. In some embodiments, digital images of a product are generated from a video recording of the product. In one example, the server 102 is configured to extract stills from a digital video file of a product to generate digital images of that product.

In some embodiments, the server 102 is configured to receive a digital image 10 of a product and (e.g., automatically, without further user intervention) execute the OCR utility 108 on the received digital image 10 to determine product content information therefrom. Determining product content information may include updating existing product content information stored on the product database 106b to include data output from the OCR utility 108. The digital images 10 may be a digital image stored on the product database 106b and associated with a specific product. In the example shown in FIG. 2, the digital image 10 is an image of a portion of a package containing a dog food and including indicia indicating ingredients thereof. In some embodiments, the system 100 may be configured to produce a scanned image file from the digital image 10 and run the OCR utility 108 on the scanned image file to determine the product content information. For example, the system 100 may be configured to generate a scanned image file of the digital image 10 and transmit the scanned image file to the server 102 to execute the OCR utility 108 thereon. In some embodiments, the OCR utility 108 includes a text similarity algorithm and matching algorithm configured to identify an exact match for predetermined attributes.

In some embodiments, the system 100 is configured to store the product content information determined via the OCR utility 108 in the product database 106b. The server 102 may be configured to receive the digital image 10 as an input to the OCR utility 108. In some embodiments, the server 102 is configured to automatically run the OCR utility 108 based on the input digital image 10 and output product content information in a machine-readable text format, such as, but not limited to: a plain text file, a comma-separated value (CSV) file, or any other suitable file type for digitally storing text. For example, the output of the OCR utility 108 based on the image 10 shown in FIG. 2 may include a text file containing a representation of the following text:
“INGREDIENTS: Ground Whole Grain Corn, Meat and Bone Meal, Soybean Meal, Animal Fat (Source of Omega 6 Fatty Acids [Preserved with BHA & Citric Acid]), Corn Gluten Meal, Natural Flavor, Dried Plain Beet Pulp, Chichen Byproduct Meal, Salt, Ground Whole Grain Wheat, Brewers Rice, Potassium Chloride, Calcium Carbonate, Choline Chloride, Dried Peas, DL-Methionine, Natural Grilled Steak Flavor, Zinc Sulfate, Yellow 6, Monocalcium Phosphate, Vitamin E Supplement, L-Tryptophan, Yellow 5, Red 40, Blue 2, Dried Carrots, Niacin Supplement (Vitamin B3), Copper Sulfate, Sodium Selenite, Potassium Iodide, D-Calcium Pantothenate (Source of Vitamin B5), Vitamin A Supplement, Riboflavin Supplement (Vitamin B2), Vitamin B12 Supplement, Thiamine Mononitrate (Vitamin B1), Vitamin D3 Supplement, Pyridoxine Hydrochloride (Vitamin B6), Folic Acid”.

The server 102 may be configured to transmit the product content information determined by the OCR utility 108 to the database 106b. In some embodiments, the system 100 may be configured to refine, parse and categorize the text included in the product content information prior to transmitting it to the product database 106b for storage. Continuing from the example above, the server 102 may be configured to detect the text “INGREDIENTS:” and categorize the text following it as ingredients of the product to which the digital image 10 corresponds. The server 102 may further be configured to parse the text following “INGREDIENTS:” based on, for example, punctuation marks appearing in said text (e.g., the commas between each individual ingredient). Still referring to the above example, the server 102 is configured to categorize the text as ingredients and parse the text based on the commas into a listing of ingredients and store on the product database 106b the categorized and parsed ingredients as product composition data having a machine-readable text format.

The server 102 may be configured to execute one or more text identification, parsing and/or categorizing algorithms on the product content information generated by the OCR utility 108. In some instances, the executed algorithm may be configured to search for specific strings of text or characters to parse the text. For example, the executed algorithm is configured to search for a string of text including a number followed by a unit of measurement (e.g., a text string of “4 oz”) to identify and parse relevant units of measure from the OCR utility output. In some instances, the executed algorithm may be configured to search for predetermined expressions in a certain format. For example, an executed algorithm is configured to search for an expression of “Warning:” or “For use with:” to parse relevant intended usage and usage restriction data from the OCR utility output. In some instances, the executed algorithm is configured to search for one or more known ingredients. In one example, the executed algorithm is configured to identify instances of the text “peanut butter” or “corn” to determine whether those ingredients are present in the product. In some embodiments, the executed algorithm is a natural language processing (NLP) algorithm trained to identify incompatibility information. In one example, the NLP algorithm may be trained to identify text that corresponds to a pet incompatibility with a product, but not limited to, text similar to: “Warning: not for use with small dogs”, “Do not feed if on heart worm medication”, “May cause drowsiness in cats under the age of 2”, and so on.

In some embodiments, OCR utility 108 is configured to detect and identifies a particular ingredient without detecting of the word INGREDIENT (or a similar indication that and ingredient listing is nearby). By detecting and identifying one ingredient, OCR utility 108 is prompted to identify other ingredients in a nearby listing. This aspect of OCR utility 108 may be useful where a package is damaged and words or phrases indicating INGREDIENTS has been adulterated.

In some embodiments, the system 100 is configured to run the OCR utility 108 on a plurality of digital image stored in the database 106b and associated with a pet product. For example, the server 102 may be configured to receive each digital image associated with a product and run the OCR utility 108 on each to determine product content information in generally the same manner as discussed above. For example, additional digital images of the packaged dog food referenced in FIG. 2 may include indicia related to serving size (e.g., oz/lb per serving for different sizes of pets), and net weight of the pet product (e.g., total oz/lb for the product). The server 102 may be configured to run the OCR utility 108 on the additional images to determine the serving size and net weight data and transmit it to the product database 106b as product content information for the product to which the digital images correspond.

Determining product content information via an OCR utility 108, as discussed above, may be beneficial in instances where there is a database of existing product information that includes digital images of the product and further product content information is desired. For example, an existing database for an online retailer may include digital images of a plurality of different foodstuffs for use at an online storefront but may be devoid of any listing of ingredients in a computer readable format. In such instances, the system 100 of the present disclosure may be implemented to significantly reduce the time required to determine such product content information and increase the accuracy with which it is determined as compared to conventional methods.

In some embodiments, the system 100 is configured to determine the accuracy of product content information determined from a digital image of a product. The server 102 is configured, in some aspects, to compare the determined product content information generated from a digital image of a product received directly from a manufacturer or vendor with product content information generated from digital image of that product located within, for example, a warehouse or other storage facility. In one example, the server 102 is configured to compare product content information determined from the different digital images of the same product and determine that the net weight values obtained therefrom are inconsistent. In some embodiments, the server 102 is configured to determine the product content information based on a hierarchy of the sources of data. Continuing from the above example, the vendor is one data source and the physical product located within the warehouse is another data source. The server 102 may attribute the physical product data source as being more trusted than the vendor data source, and therefore above the vendor on the data source hierarchy. In some instances, the server 102 is configured to assign the net weight value from the physical product data source as the net weight value included in the product content information. In some embodiments, in response to determining an inconsistency for determined product content information, the server 102 is configured to flag the product for a manual review and input of the correct product content information.

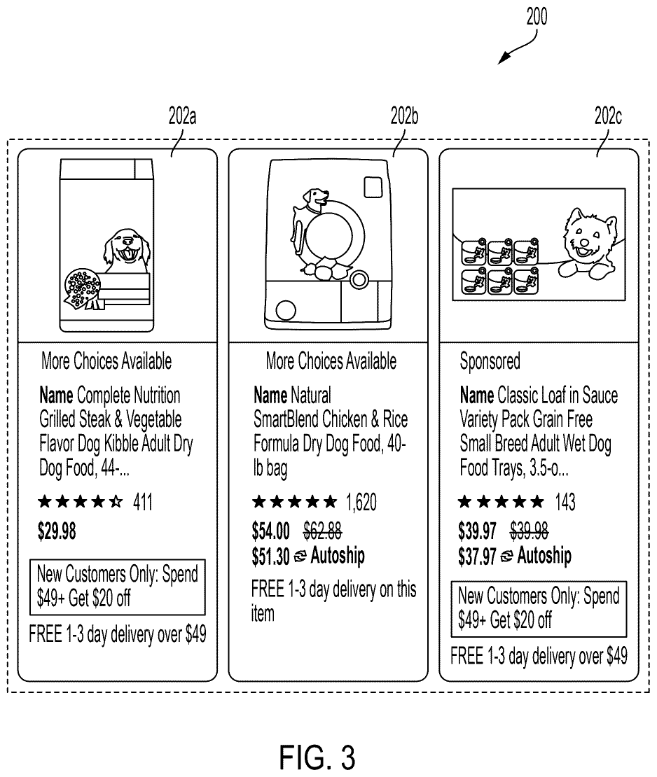

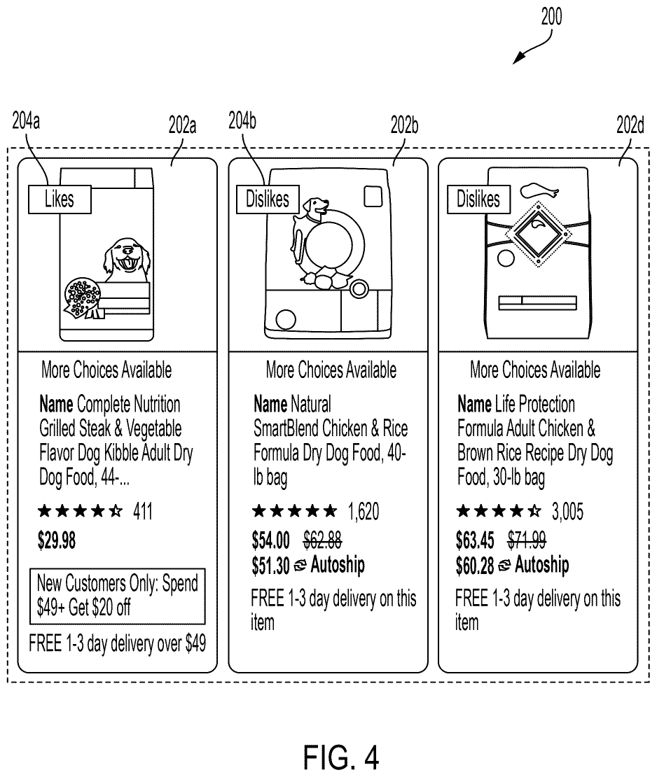

Referring to FIGS. 3-4, in some embodiments, the system 100 is configured to cause a UI 200 to render information associated with a pet-product relationship at products displayed at the UI 200. The UI 200 may include one or more fields or graphical elements (e.g., interactable graphical elements) corresponding to different products and/or services. For example, and as shown in FIGS. 3-4, the UI 200 shown is a portion of a UI for an online storefront offering for sale different dog foods. In FIG. 3, there are three products 202a-202c displayed at the UI 200. Products as referenced in relation to the UI 200 may refer to an interactable field and/or digital display of a corresponding product. For sake of brevity, aspects of the present disclosure will be described in reference to food products displayed at the UI 200 and pet-product relationship data corresponding to those products. However, the system 100 may be configured to determine pet-product relationship data for any good and/or service (e.g., ads for pet daycare services, pet grooming services, dog walking services). In some embodiments, the products 202a-202c may be offered for sale at the UI 200. In some embodiments, the UI 200 may display information for various products while providing an offer for sale thereof. The storefront server 112 may be configured to generate the UI 200 and transmit the UI 200 to a client device 110 for display thereon via network 111. The UI 200 illustrated in FIGS. 3-4 may be rendered at a client device 110.

The system 100 may be configured to compare the product content information associated with a product with pet information stored in the pet profile database 106a to generate the pet-product relationship data. For example, in response to the UI 200 being displayed at a client device 110, the server 102 may be configured to query the product database 106b for product content information associated with the products 202a-202c. In some embodiments, the server 102 is configured to generate pet-product relationship data in response to a request to display the UI 200 at a client device 100. For example, prior to the UI 200 being displayed at the client device 110 the server 102 may receive a request to display the UI 200 and automatically generate the pet-product relationship data.

The server 102 may be configured to query the pet profile database 106a for pet information associated with the user's pet and compare the queried product content information to the queried pet information to generate pet-product relationship data. The pet-product relationship data may include data indicative of an expected reaction by the pet in response to interaction with a product. An expected reaction may include, but is not limited to, physiological responses (e.g., an allergic reaction, vomiting), and/or psychological responses (e.g., behavior reactions indicating discomfort, disinterest, or the opposite). For example, the server 102 compares the product composition data (e.g., listing of ingredients) stored on product database 106b for each of products 202a-202c to the pet allergy data and dietary preference data stored on the pet profile database 106a to determine pet-product relationship data. Further to this example, the pet-product relationship data determined by server 102 for the user's pet Chopper and products 202a-202c includes data indicative that the pet Chopper is expected to like the product 202a, dislike the product 202b and experience an allergic reaction to the product 202c.

In some instances, the pet-product relationship data is based on, at least in part, a previous purchases of a product. In one example, the server 102 is configured to generate pet-product relationship data indicating that a user's pet may be tired of a particular foodstuff based on purchase data indicating that the particular foodstuff has been repeatedly purchased over a long period of time (e.g., at least six months, at least one year). In some instances, in response to determining pet-product relationship data indicating that a user's pet is likely to be tired of a particular pet product, the server 102 may be configured to identify alternative products thereto and include a recommendation of those products in the pet-product relationship data. For example, if the server 102 determines that the user's pet is likely to be tired of eating pet food A, the server 102 is configured to identify pet food B as an alternative thereto and include a recommendation of pet food B in the pet-product relationship data, which may be displayed to the user as discussed in more detail below.

In some instances, the server 102 is configured to generate the pet-product relationship data based on ingredients of the corresponding products. For example, and continuing from the above example, the pet information for Chopper may include that Chopper prefers dog foods with steak, dislikes dog foods containing chicken and is allergic to grain free dog foods. The product information for products 202a-202c may indicate that the ingredients of product 202a include steak, the ingredients of product 202b include chicken, and the ingredients of product 202c do not include any type of grain. The server 102 may be configured to determine the pet-product relationship data based on the pet information for Chopper and the product content information for products 202a-202c that the user's pet Chopper may like product 202a, dislike product 202c, and may be allergic to product 202c.

In some embodiments, the system 100 is configured to direct the UI 200 to render information associated with the determined pet-product relationship data. The rendered information may be, in some instances, a visual representation including text, colors, and/or symbols indicating the pet-product relationship data for products displayed at the UI 200. For example, and as shown in FIG. 4, the server 102 may be configured to direct the storefront server 112 to render informational GUI elements 204a-204b at the products 202a-202b. The GUI elements 204a-204b appear on the UI 200 as banners with text indicating whether the user's pet Chopper likes or dislikes the corresponding product. For example, the GUI element 204a includes a rendering that Chopper likes the product 202a and the GUI element 204b includes a rendering that Chopper dislikes the product 202b. The GUI elements 204a-204b appearing in FIG. 4 are for illustrative purposes only and it should be understood that a plurality of different visual indications or combinations thereof may be used.

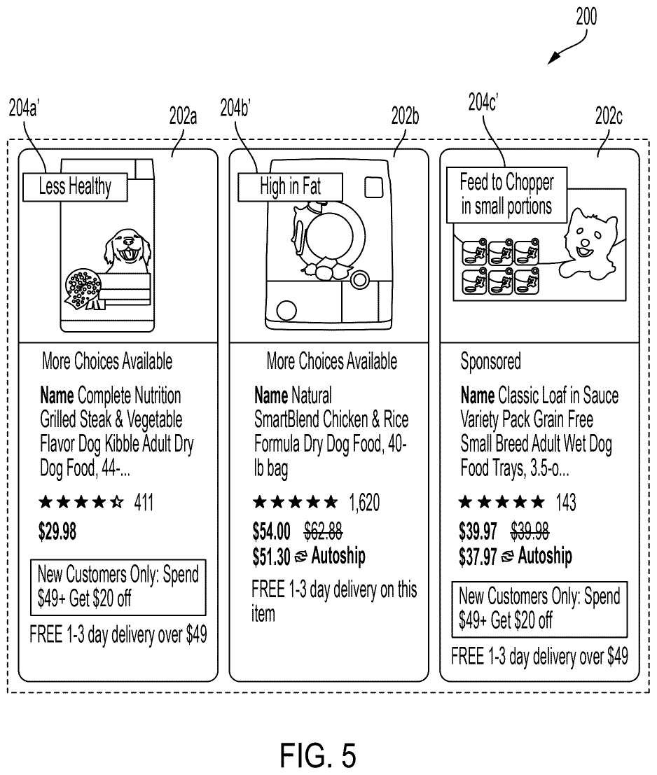

The system 100 may be configured to enable the user to quickly and easily discern pet-product relationship data for their particular pet via the rendered information (e.g., GUI elements 204a-204b). This may be particularly beneficial in instances where a plurality of products are displayed simultaneously, each product being comprised of a plurality of different ingredients, and the user must consider different factors specific to their pet when selecting from those products. The example illustrated in FIGS. 3-4 demonstrate rendering information associated with the pet-product relationship data for a pet's liked, disliked, and allergic food ingredients. However, the system 100 may be configured to determine pet-product relationship data based on a plurality of different types of pet information and product content information stored on the respective databases 106a, 106b. The system 100 may be configured to determine the pet-product relationship data by comparing 1) pet information such as, but not limited to: pet age, pet breed, pet height, pet weight, pet allergies, pet likes, pet dislikes, pet medical history or a combination thereof to 2) product content information such as, but not limited to: product ingredients, product serving size, product weight, product intended use, calories, or a combination thereof. In one embodiment, indication of a particular food that is approved or rejected for a specific pet may be indicated based on the breed of the pet. For example, if Chopper is a breed of dog that is known to be relatively inactive, food that is high in fat may be less healthy and the UI 200 might indicate it should be fed to Chopper only in small portions or not at all.) For example, and referring briefly to FIG. 5, the UI 200 may include a GUI elements 204a′-204c′ including different variations of text displays providing a visual indication to the pet owner that the product 202 is high in fat and should be fed to Chopper in small portions.

As another example, and referring to FIGS. 3-4, product content information may include that a particular dog food product displayed at the UI 200 is intended for small dog breeds between 10-20 pounds in weight and the pet information for a user's pet may indicate that the pet is an adult rottweiler weighing 90 pounds. The system 100 may be configured to determine that the displayed small breed dog food product is unintended for, or unsuitable for the user's pet. The system 100 may be configured to direct the UI 200 to render information associated with that determination (e.g., the pet-product relationship data) at the product displayed at the UI 200. For example, the server 102 may direct the UI 200 to render a banner with the text “to large for” or “intended for smaller dogs” appearing on or over the small dog food product similar to what is shown in FIG. 4.

In some embodiments, the system 100 is configured to automatically remove renderings of products at the UI 200 that are expected to be harmful to or have an adverse effect on a user's pet based on the pet-product relationship data. In some embodiments, a product that is determined to be harmful to or have an adverse effect on a user's pet may be a product including an ingredient that the pet is allergic to. As discussed above, the pet profile information stored in the pet database 106a may include a listing of foodstuffs, ingredients thereof, or other substances that the pet is allergic to. In response to a user having pet profile information stored on the pet database 106a rendering the UI 200 on their respective client device 110, the system 100 may automatically determine pet-product relationship data and direct the UI 200 to replace a rendering of an allergic or toxic pet product with one that is not. For example, FIG. 3 illustrates the UI 200 prior to determining pet-product relationship data and as such, the product 202c is rendered thereon.

The server 102 may determine the pet-product relationship data for the user's pet Chopper, from the previous example, which may include a determination that Chopper is allergic to an ingredient of product 202c. The server 102 may be configured to automatically cause the UI 200 to replace the rendering of product 202c with a rendering of another product 202d, to which the pet Chopper is not allergic (as shown in FIG. 4). The system 100 of the present disclosure may automatically determine the pet-product relationship data and automatically filter out, or remove, a plurality of products rendered at the UI 200 which may have an adverse effect on the user's pet. This may be particularly beneficial to users because the system 100 is configured to significantly reduce the time required to do such filtering manually and improves the accuracy with which it is accomplished.

In some embodiments, the pet-product relationship data comprises at least one of breed-specific pet information, allergy information for a specific pet, ingredient compatibility information with a specific pet, product compatibility information with a specific pet, ingredient dislike information for a specific pet, and product dislike information for a specific pet. Breed-specific pet information may include, for example, if the pet's breed is a Boxer that a product with high grain content is at an increased likelihood to cause an allergic reaction. Allergy information for a specific pet may include that, for example, the user′ pet Chopper is allergic to soy. Ingredient compatibility information may include, for example, that certain ingredients a preferred by the pet, and/or that certain ingredients may cause the pet to have increased bowel movements. Product compatibility information may include, for example, that a pet has had a certain reaction to a specific product in the past (e.g., Product A makes the pet energetic, Product B makes the pet tired). Ingredient dislike information and product dislike information may generally include information relating to whether a pet dislikes a product in its entirety or if there is an ingredient in a product that the pet dislikes.

The system 100 may be configured to automatically determine a listing of allergic substances based on the pet breed and/or type information included in the pet profile information.

For example, in an instance where the pet profile information indicates that a user's pet is a breed of dog, the system 100 may be configured to automatically remove any renderings at the UI 200 of products that include chocolate, being that chocolate is toxic to dogs. As a further example, pet profile information may include that a user's pet is a male Boxer, which is a breed particularly sensitive to high grain content (e.g., corn, wheat) foodstuffs. The system 100 may be configured to automatically determine that pet-product relationship data for pet products containing corn and wheat is likely to have an adverse affect on the user's Boxer and may direct the UI 200 to either 1) remove renderings of products having high grain content or 2) render an indication that the user's pet may be allergic (e.g., a banner similar to GUI element 204b that includes the text “may be allergic”).

Still referring to FIGS. 3-4, in the above examples, the user accessing an instance of the UI 200 at their client device 110 (e.g., as shown in FIG. 4) causes the system 100 to automatically associate that instance of the UI 200 with the pet profile information associated with that user. The user may have associated user account specific to the online storefront and stored on a database in communication with the server 102 and/or storefront server 112. The user account may be, in some instances, associated with a single pet profile stored on the pet database 106a. The system 100 may be configured to receive an indication of the user's account in relation to the UI 200 being rendered at a client device and automatically associate that instance of the UI 200 with that user account and the associated pet profile. For example, the system 100 may detect login credentials or another form of identification (e.g., a user logging in at the UI 200 to their user account) at the UI 200 and automatically associate that instance of the UI 200 with the user account and the associated pet profile information. In response to associating the instance of the UI 200 with pet profile information, the system 100 may be configured to automatically determine pet-product relationship data and direct the UI 200 to render information associated therewith as discussed above.

In other instances, the user account may be associated with two or more pet profiles and the system 100 may be configured to determine which of the pet profiles to determine pet-product relationship data for. A user may own two pets (e.g., a male rottweiler and a female goldendoodle), and in response to the user logging in to their user account at the UI 200, the system 100 may be configured to direct the UI 200 to render a request for a selection of one of the two pets. For example, the server 102 may direct the UI 200 to render, at the client device 110, a modal window with one or more interactable fields requesting a selection of either the male rottweiler or the female goldendoodle. In response to detecting a selection of one of the two or more pets (e.g., a selection of the goldendoodle) the system 100 may automatically determine pet-product relationship data for the selected pet's associated pet profile information in generally the same manner as discussed above.

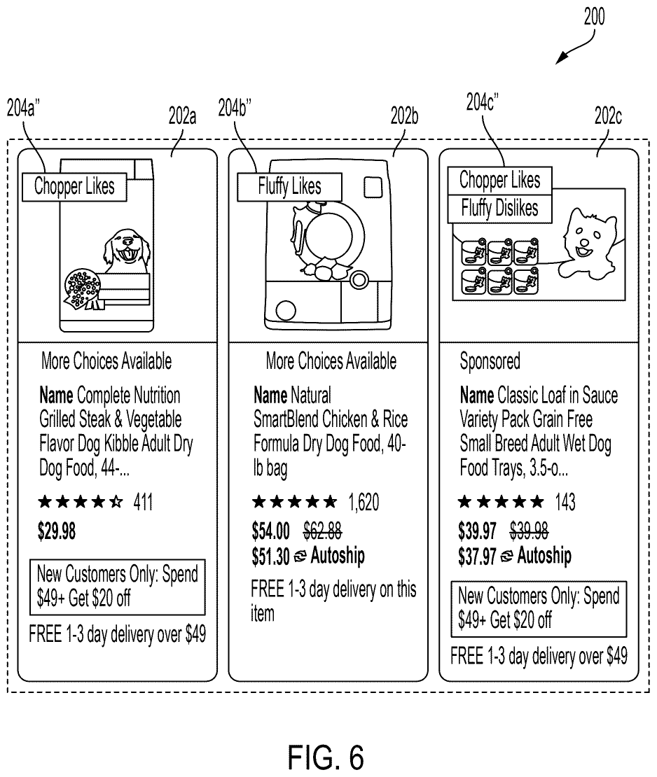

Referring to FIG. 6, in some instances, the user account may be associated with two or more pet profiles and the system 100 may be configured to direct the UI 200 to render pet-product relationship data for each pet profile simultaneously. For example, the user account may be associated with pet profile information for a rottweiler named Chopper and a goldendoodle named Fluffy. The system 100 may be configured to determine pet-product relationship data for both Chopper and Fluffy and direct the UI 200 to display a rendering of the pet-product relationship data. The server 102 may be configured to direct the UI 200 to render two GUI elements 204 for at least one product 202 indicating the name of the pet and information about the associated pet-profile relationship data. Continuing from the above example, a GUI elements 204 for a product displayed at the UI 200 may include the text “Chopper likes” and the other may include text “Fluffy dislikes”. Further to this example, and as shown in FIG. 6, the GUI elements 204c″ include an indication of the pet-relationship data for Chopper and Fluffy with regards to product 202c, while the GUI elements 204a″ and 204b″ include an indication of the pet-relationship data for Chopper and Fluffy with regards to products 202a-202b respectively. In other instances, the system 100 may be configured to render a single GUI element 204 including information associated with the pet-product relationship data for each pet (e.g., a single GUI element 204 with the text from the previous example).

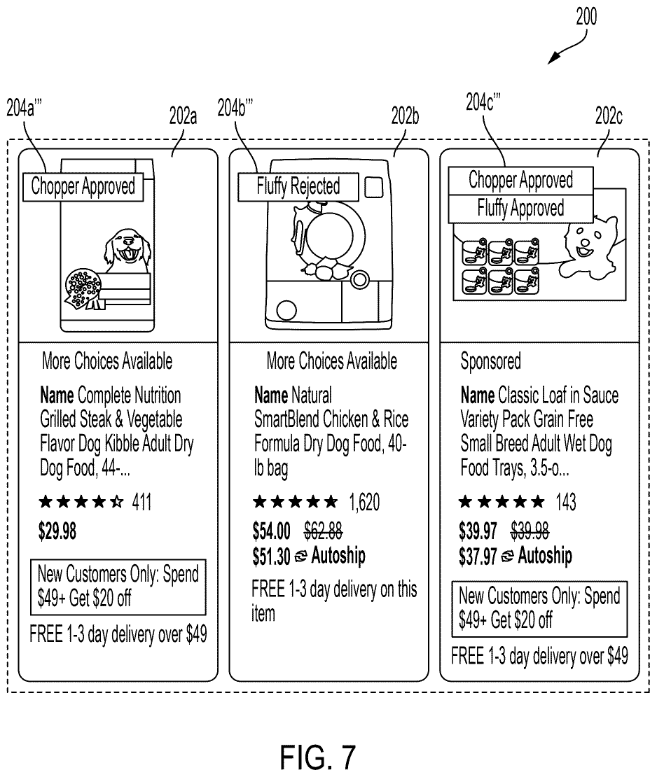

Referring to FIG. 7, in one embodiment, upon a user logging into the storefront and reviewing the UI 200 displaying products 202a, 202b, 202c, the UI 200 may further render a graphic illustration of which of the user's multiple pets are candidates for the product being displayed. For example, product 202a may include the GUI element 204a″′ indicating that product 202a is a CHOPPER APPROVED product. In one embodiment, UI 200 is rendered to illustrate more than one pet's likes and dislikes. For example, product 202c may include GUI element 204c″′ indicating that the product 202c is CHOPPER APPROVED and that same product is FLUFFY REJECTED. In one rendered UI 200 illustrates a product as CHOPPER APPROVED and FLUFFY APPROVED. In one rendered UI 200 there may be illustrated a first product that is CHOPPER APPROVED and a second product as FLUFFY APPROVED as illustrated in, for example, GUI elements 204a″′ and 204b″′. In some instances, the system 100 is configured to automatically determine pet-product relationship data to render at the UI 200 for different pet profile information based on one or more differences in the pet profile information. As a non-limiting example, a difference between pet profile information for two different pets may include: that each pet is a different type of animal (e.g., a cat and a dog), an age difference between the two pets (e.g., a 6-month old puppy and a 7 year old dog), a difference in weight between the two pets, a difference in breed (e.g., a rottweiler and a goldendoodle), a difference in height, a difference in dietary restrictions, or any combination thereof. In some embodiments, the system 100 may be configured to determine pet-product relationship data for two or more pets and direct the UI 200 to render information associated with each based on a best match to the product displayed at the UI 200. For example, the system 100 may determine pet-product relationship data for both a cat and a dog. In an instance where the UI 200 includes a plurality of products for cats and a plurality of products for dogs simultaneously, the server 102 may be configured to direct the UI 200 to render pet-product relationship data for the dog at the dog products and pet-product relationship data for the cat at the cat products.

In some embodiments, a logged in user may be reviewing products that are incompatible with a pet identified in the user's profile. UI 200 may be configured to prompt the user to update their profile to include the pet that is compatible with the product. In some instances, the prompt may include a possible pet breed that is consistent with the product for the user to select when updating the profile. In some embodiments, the graphical user interface includes a toggle utility that allows a user to toggle (e.g., via touch screen) between displays that show only pet-specific compatible products with displays that show both pet-specific incompatible products and pet-specific compatible products.

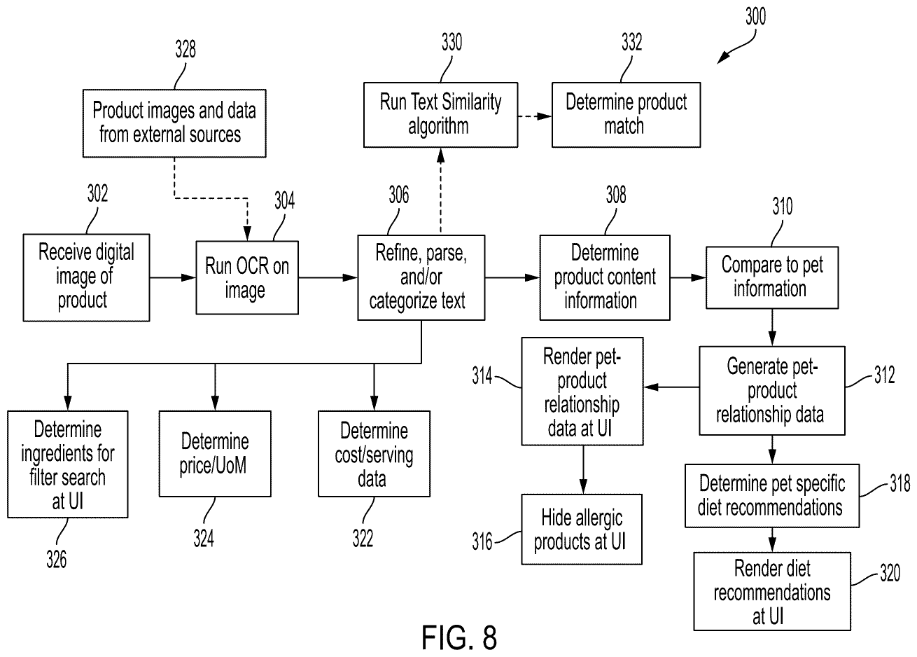

Referring to FIG. 8, there is illustrated an exemplary flow chart for a method of matching a pet to a pet product, generally referred to as method 300. In some embodiments, the method 300 is executed by the system 100 as described above. In some embodiments, the method may include the step 302 of receiving a digital image of a product. For example, and as discussed above with reference to FIG. 2, the system 100 may be configured to receive a digital image 10 of a product, which in some instances may include producing a scanned image file from a digital image of the product. In some embodiments, the method 300 may include the step 304 of running an optical character recognition utility on the scanned image file. For example, and as shown in FIG. 2, the server 102 includes an OCR utility 108 configured to run on the digital image 10 to produce a text file to be stored in the product database 106b. In some embodiments, the method 300 may include the step 306 of refining, parsing, and/or categorizing the text output from the OCR utility 108. For example, and as discussed above with reference to FIG. 2, the server 102 is configured to categorize the text from the digital image 10 as ingredients of the product and parse the text to determine each ingredient included therein. In some embodiments, the steps 304 and 306 may be combined into a single step. The steps 302-306 may be repeated for each digital image of a product and for a plurality of different products.

The method 300 may include the step 308 of determining product content information. For example, and as show in FIG. 2, the server 102 is configured to transmit the output from the OCR utility 108 from steps 304-306 to the product database 106b as product content information. In some embodiments, the method 300 may include the step 310 of comparing the product content information associated with the product with pet information stored in a pet profile database. For example, and as discussed above, the system 100 is configured to compare the product content information stored on the product database 106b with pet information stored on the pet profile database 106a. In some embodiments, the product content information referenced in step 310 is the product content information determined in step 308. In other instances, the product content information referenced in step 310 is from another source (e.g., a direct upload from a manufacturer of the product).

In some embodiments, the method 300 includes the step 312 of generating pet-product relationship data. For example, the system 100 may be configured to, based on the comparison (e.g., from step 310) generate pet-product relationship data as discussed above. The pet-product relationship data may include, for example, information related to whether a pet likes, dislikes, or is allergic to a product. For example, pet-product relationship data for a pet and a product may include information that the pet likes that product, and so on for a plurality of products.

In some embodiments, the method 300 includes the step 314 of directing a user interface to render information associated with the pet-product relationship data. For example, and as shown in FIG. 4, the server 102 may be configured to direct the UI 200 to render the GUI elements 204a, 204b, and 204d each one including information associated the pet-product relationship data for a pet and the respective products 202a, 202b, and 202d. In some embodiments, the method 300 may include a step 316 of preventing the user interface from displaying product purchase information associated with the product based on an identified mismatch data. For example, the system 100 is configured to determine whether pet-product relationship data for a specific product includes information that the pet is allergic to that product. In response to that determination, the system 100 may direct the UI 200 to replace the rendering of that allergic product with another product, as shown in FIGS. 3-4 and as discussed above.

In some embodiments, the method 300 includes identifying mismatch data based upon a request received via the user interface to display product purchase information based on a product query and the pet-product relationship data and restricting display of at least one product based on the mismatch data. The system 100 may be configured to detect request to display products at the UI 200 matching a selection and/or input of a product query (e.g., dog food products under $50.00) at the UI 200. The system 100 may, in response to detecting the request, be configured to display at least one product based on the pet-product relationship data. For example, and as shown in FIGS. 3-4, the system 100 may detect a request to display dog foods and in response to detecting that request, determine the pet-product relationship data for products 202a-202c, identify that there is a mismatch with product 202c, and display at the UI 200 products 202a-202b and 202d.

In some embodiments, the method 300 may include the step 318 of determining pet specific diet recommendation data. The system 100 may be configured to determine diet recommendation data based on the pet-product relationship data. Diet recommendation data may include, but is not limited to, indications of products for weight loss, products for weight gain, products for improved gastrointestinal health, products to improve dental health, products to improve energy, or any combination thereof. For example, pet information stored on the pet database 106a may indicate that a pet is overweight for its breed, age, and/or size. The system 100 may be configured to determine that pet-product relationship data for a pet foodstuff that is higher in calories than other similar foodstuffs is less recommended should the pet owner desire to reduce the pets weight. The method 300 may include the step 320 of directing the UI 200 to display the diet recommendation data. For example, the system 100 may be configured to direct the UI 200 to display at a GUI element 204 for one or more products 202 a text indication such as “High in calories” or “recommended for weight loss”.

The method 300 may include the step 322 of determining a cost per serving value for products and displaying that value at the UI 200. The system 100 may be configured to determine the cost of a product in relation to a standard or recommended serving size based on the output from the OCR utility 108. For example, a digital image of a product may include a suggested serving size as printed on a portion of the package the product is contained in (e.g., 1 cup of dog food per day for dogs up to 10 lbs, 1.75 cups for dogs between 10 to 25 pounds, and so on). The OCR utility 108 may receive the digital image, convert it to text data and refine the text data as described in relation to steps 302-306 to determine serving size information. The system 100 may be configured to compare that serving size information to the pet information stored in the pet profile database 106a (e.g., the pet weight data), the product information stored in the product database 106b (e.g., product cost data, product volume data, product weight data) and determine the price per serving size. For example, the server 102 may determine that the appropriate serving size for a pet food product is 1 cup for a user's pet that weighs 7 pounds. Based on the determined serving size, the server 102 may further calculate the total number of cups of the pet food product included in a single purchase of that food product and determine the cost of the product to calculate the cost per serving value.

Continuing from the preceding example, a pet food product containing 50 cups of foodstuffs that costs $50.00 would have a cost per serving size value of $1.00 per serving. The system 100 may be configured to direct the UI 200 to render the determined price per serving size value for products 202 displayed thereon. The system 100 may be configured to automatically determine the cost per serving size value for a plurality of different products based on a user's pet and display that value at the UI 200 thereby providing users with personalized and detailed product information that is not contained within the digital image of the product.

In some embodiments, the method 300 may include the step 324 of determining and displaying at the UI 200, a price per unit of measure (UoM) for products 202 displayed thereon. The step 324 may be generally the same as the step 322 discussed above except that the determined value is based on a UoM (e.g., pounds, ounces) instead of a recommended serving size. For example, the system 100 may be configured to determine from a digital image of the product (e.g., as outlined in steps 302-306) that a product contains 60 pounds of foodstuff. The system 100 may further determine from the product information stored on the product database 106b that the price of that product is $30.00 resulting in the price per UoM, which in this instance is pounds, is $0.50 per pound. The system 100 may be configured to direct the UI 200 to render an indication of the price per pound (e.g., a GUI element displaying the price per pound as text).

In some embodiments, the method 300 may include directing the UI to display unit price comparison data for all products responsive to a product query based on the pet-product relationship data. For example, the system 100 may be configured to determine the unit price of each product matching a product query (e.g., all dry dog foods) and direct the UI 200 to render text indicating that unit price at each product displayed thereon matching the product query. In some embodiments, the unit price comparison data may include a comparison of the determined price per serving value (e.g., from step 322) and/or the determined price per UoM value (e.g., from step 324).

In some embodiments, the method 300 may include the step 326 of determining a listing of ingredients for products displayed at the UI 200 and rendering an interactable ingredient filter at the UI 200. The system 100 may be configured to determine a listing of ingredients for products 202 displayed at the UI 200 based on digital images of those products as discussed above with regards to FIG. 2. Based on the determined listing of ingredients, the system 100 may be configured to direct the UI 200 to display an interactable filter search menu to enable a user to select from one or more ingredients with which they wish to refine their search of products. For example, the interactable filter search menu may include a listing of ingredients such as, but not limited to: whole grain corn, salt, and brewers rice. The system 100 may detect a selection of whole grain corn and direct the UI 200 to only render products 202 containing whole grain corn as an ingredient. In some instances, the filter search menu may be configured to enable a user to select from ingredients they wish to avoid and the system 100 may be configured to direct the UI 200 to remove renderings of products 202 containing those ingredients.

In some embodiments, system 100 is configured to display a listing of ingredients in a manner that highlights (e.g., in contrasting text), in the list of ingredients, those ingredients that may be of interest to the user, based on stored pet data. For example, if pet data indicates that a pet has a distaste or allergy response to a particular ingredient, that ingredient may be highlighted in the list of ingredients. In some embodiments, system 100 is configured such that a user can hoover a mouse over a highlighted ingredient to obtain information as to why the particular item is highlighted in the list of ingredients.

In some embodiments, the method 300 may include receiving a product request, via the user interface, to display available products, and determining that the product request is not associated with a particular pet. For example, the system 100 is configured to determine, in response to displaying an instance of the UI 200 at a client device 110 that there is no pet information associated with the user's account. In some instances, the system 100 is configured to determine there is no pet information in response to a request to display products available for purchase at the UI 200 (e.g., a selection of dog foods for sale). In some embodiments, the method 300 further includes, prior to rendering, on the user interface, product information based on the product query, directing the user interface to render a pet information request. For example, and as discussed above, the system 100 may be configured to direct the UI 200 to render a modal window with one or more input fields requesting a selection of, or information pertaining to the user's pet. A modal window is intended as a non-limiting example and any other suitable form of rendering a request for information at a UI may be used. In some embodiments, the requested information may include, but is not limited to: pet name, pet age, pet weight, pet breed, pet type, pet allergies, genetic diseases, health history based on a family tree, pet home history (e.g., how many times the pet has been homed by different owners) or any combination thereof.

The method 300 may further include receiving, via the UI, pet identification information based on the pet information request, identifying pet product relationship data based on the product query and the received pet identification information, and directing the UI to display product information based on the product query and the pet-product relationship data. For example, the system 100 may be configured to receive the requested pet information and determine pet-product relationship data in generally the same manner as discussed above. The determined pet-product relationship data may be specific to the products requested for purchase (e.g., dry dog foods). The system 100 may, in response to determining the pet-product relationship data, direct the UI 200 to display information thereon in generally the same manner as described with reference to FIG. 4.

In some embodiments, the method 300 may include, applying to a product package prior to shipment by a fulfilment center, a pet specific label based on the pet-product relationship data. For example, in some instances, the system 100 may include a fulfillment center (FC) server in communication with the network 111 and the server 102. The FC server may be in communication with an applicator for applying shipping labels to a packaged product (e.g., a label making device including an applicator). In response to a purchase of a product (e.g., product 202a in FIG. 4) from the UI 200, the server 102 may generate a digital record of the order including a unique order identifier and the pet-product relationship data for the product that was purchased. The server 102 may be configured to cause the digital record of the order to be transmitted to the FC server at or before the time at which the order is to be packed and labels (e.g., shipping labels) applied thereto. In response to receiving the digital record, the FC server may be configured to direct the applicator to print a label including text specific to the pet-product relationship data (e.g., “Chopper really likes the steak in this product”) and apply it to a shipping container (e.g., shipping box) containing the product. In some instances, the label making device may be configured print a packing slip or sheet of parchment to be packed inside of a shipping container that includes, for example, an image of the pet and the product and text specific to the pet-product relationship data. In some instances, the label may include a QR code or other indicia that when accessed by an authorized user device associate with a pet, displays on the user device information about the contents of package and preferences associated with the pet associated with the order.

In some embodiments, the information associated with the pet-product relationship data includes product recommendation information. The system 100 may be configured to identify product to which a user's pet may like based on the pets likes and/or dislikes included in the pet information stored on pet profile database 106a. For example, if pet information includes that the pet likes salmon, then the system 100 may be configured to automatically determine which pet foodstuffs contain salmon as an ingredient and automatically direct the UI 200 to render that information on those products displayed thereon. In some embodiments, the method 300 may include directing the user interface to display a supplied product image and product content information that is not displayed on the supplied product image. For example, the system 100 may be configured to display an image of the foodstuff product 202a at the UI 200 which is devoid of any price per pound information.

In some embodiments, the method 300 may include matching products offered for sale at the UI 200 to products offered for sale from sources external to the UI 200. The method 300 may include the step 328 of receiving product images and/or data of products offered for sale from external sources. In some instances, external sources may include, but are not limited to online storefronts, or other retail sources, other than the online storefront generated by the storefront server 112. The digital images received at step 328 may be provided as input to the OCR utility 108 and the output of which may be refined, parsed, and/or categorized in generally the same manner as discussed above with regards to steps 304-306. For example, the system 100 may be configured to receive digital images of products offered for sale at external sources and determine product content information in generally the same manner as discussed above with regards to FIGS. 1-2 and steps 302-308 of the method 300.

In some embodiments, the method 300 includes the step 330 of running a text similarity algorithm on text output from the OCR utility 108 for products offered for sale at the UI 200 and product content information for products offered for sale at external sources. For example, the system 100 may be configured to execute a text similarity algorithm on the refined, parsed and/or categorized text for products 202 and externally sourced products to compare the similarity between them. In some instances, comparing the text similarity includes comparing similarity of text indicating product attributes such as, but not limited to: product net weight, product ingredients, and/or product calories. The output from the similarity algorithm may be used to determine matches between products. In some embodiments, the method 300 may include the step 332 of determining a product match between a product offered for sale at UI 200 and an externally sourced product. For example, the system 100 may be configured to determine a product 202 matching an externally sourced product based on the output of the text similarity algorithm from step 330.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways.

Specific features of the exemplary embodiments may or may not be part of the claimed invention and one or more features of the disclosed embodiments may be combined in whole or in part. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

1. A method of matching a pet with a pet product comprising:
at a server system including one or more processors:
generating pet-product relationship data by comparing product content information associated with a product to pet information stored in a pet profile database; and
causing a user interface to update a rendering of the product to include a rendering of information associated with the pet-product relationship data.

2. The method of claim 1 further comprising:
at the server system including one or more processors:
accessing a scanned image file from a digital image of a product; and
running an optical character recognition utility on the scanned image file to determine the product content information associated with the product,
wherein the comparing the product content information associated with a product with pet information is based on the determined product content information.

3. The method of claim 1 further comprising:
at the server system including one or more processors:
preventing the user interface from displaying product purchase information associated with the product based on the pet-product relationship data.

4. The method of claim 1 further comprising:
identifying mismatch data in response to receiving a request via the user interface to display product purchase information based on a product query and the pet-product relationship data; and
restricting display of at least one product based on the mismatch data.

5. The method of claim 1 further comprising:
at the server system including one or more processors:
receiving a product request, via the user interface, to display available products based on a product query;
determining that the product request is not associated with a particular pet;
prior to rendering, on the user interface, product information based on the product query, directing the user interface to render a pet information request;
receiving, via the user interface, pet identification information based on the pet information request;
based on the product query and the pet identification information, identifying pet-product relationship data; and
directing the user interface to display product information based on the product query and the pet-product relationship data.

6. The method of claim 1 further comprising:
at the server system including one or more processors:
causing an applicator device to apply a pet-specific label to a product package prior to shipment by a fulfillment center,
wherein the pet-specific label is based on the pet-product relationship data.

7. The method of any of claim 1, wherein the information associated with the pet-product relationship data includes product recommendation information.

8. The method of claim 1, wherein the pet-product relationship data comprises at least one of breed-specific pet information; allergy information for a specific pet; ingredient compatibility information with a specific pet; product compatibility information with a specific pet; ingredient dislike information for a specific pet; and product dislike information for a specific pet.

9. The method of claim 1, wherein the product content information includes at least one of product ingredient information; product allergy information; unity price information; and information not contained within product package indicia.

10. The method of claim 2, wherein the optical character recognition utility includes a text similarity algorithm and matching algorithm configured to identify an exact match for predetermined attributes.

11. The method of claim 1 further comprising:
at the server system including one or more processors:
directing the user interface to display a supplied product image and product content information that is not displayed on the supplied product image.

12. The method of claim 1 further comprising:
at the server system including one or more processors:
directing the user interface to display unit price comparison data for all products responsive to a product query based on the pet-product relationship data.

13. The method of claim 1 further comprising:
at the server system including one or more processors:
directing the user interface to display diet recommendation information based on the pet-product relationship data.

14. The method of claim 1, wherein the product content information includes a parsed list of ingredients corresponding to the product and the pet information includes pet allergy data and pet dietary preference data, and
wherein the pet-product relationship data is indicative of an expected interaction with the product based on a comparison of the parsed list of ingredients to the pet allergy data and pet dietary preference data.

15. A system including one or more processors and memory storing one or more programs to be executed by the one or more processors, the one or more programs including instructions for executing one or more of the operations included in claim 1.

16. A non-transitory computer readable storage medium storing one or more programs configured for execution by a computer system, the one or more programs including instructions for executing one or more of the operations included in claim 1.
